(19) United States
(12) Patent Application Publication
(10) Pub. No.: US 2011/0064826 A1
(43) Pub. Date: Mar. 17, 2011

(54) COMPOSITION FOR TREATING SKIN LESIONS

(76) Inventor: John Spurge, Mareeba (AU)

(21) Appl. No.: 12/633,599

(22) Filed: Dec. 8, 2009

Related U.S. Application Data
(63) Continuation-in-part of application No. 11/993,236, filed on May 6, 2010.

(30) Foreign Application Priority Data
Jun. 20, 2005 (AU) 2005903229

(51) Int. Cl. A61K 33/34; A61K 36/38; A61P 17/00; A61P 17/02; A61P 17/16

(52) U.S. Cl. 424/632; 424/630; 424/637; 424/730; 424/638

(57) ABSTRACT

The present invention provides a composition for topical treatment of skin and mucosal membrane lesions comprising a synergistic combination of copper compound and hypericum perforatum extract.

### RELATED APPLICATION DATA

This application is a continuation-in-part of U.S. patent application Ser. No. 11/993,236, now pending, which is a 371 National Phase Application of PCT/AU2006/000863, filed Jun. 20, 2006, which claims priority benefit of Australian Patent Application No. 2005903229, filed Jun. 20, 2005. The contents of each of the foregoing applications is hereby incorporated in its entirety by reference.

### TECHNICAL FIELD

The present invention relates to a composition for the treatment of skin lesions and use of the composition. In particular, the invention relates to compositions for treatment of skin lesions associated with viral infections, such as Herpes simplex.

### BACKGROUND ART

Herpes is a sexually transmitted disease (STD) caused by the herpes simplex virus (HSV). There are two types of HSV; Herpes Simplex 1 (HSV1) and Herpes Simplex 2 (HSV2).

HSV1 or mouth herpes is commonly in the form of cold sores on and around the mouth. HSV2 or genital herpes is a much more intense strain commonly found on the genitals. However both types can be found on the mouth or genital areas. It is possible to be infected by both HSV1 and HSV2. Being infected by one particular strain does not make you immune to another.

Recurrent outbreaks of the Herpes virus generally follow a staged progression. The stages are easily identifiable and include prodrome, vesicles, ulceration, crust and healing. Prodrome is generally a short period of tingling, itching, numbness or burning with no visible sign of an outbreak. Vesicles are the formation of one or more fluid-filled blisters, often in a cluster and usually surrounded by sore, red skin. The ulceration stage is when the blisters open to form painful ulcers or open sores. At the edge of the sore, a soft or hard yellow crust begins to appear. Ulcers and painful, sore, red skin persist through this stage. At the crust stage, weeping sores or ulcers become completely covered by a crust or scab. No ulcers or blisters are present. The healing process is manifested by disappearance of the crust, swelling, pain and itching. Skin eruptions due to viral infection, especially Herpes viruses, generally have a normal infective course that lasts from 10 to 60 days depending on the exact causative species and anatomical location of the infection.

After the initial outbreak, the virus usually lies dormant in the skin or in nerve tissue (latent state) until something triggers another eruption or site infection. When the virus reactivates, it characteristically causes a sore at the site where it first entered the body. Often the trigger is unknown, but in some people overexposure to sunlight, fever, physical or emotional stress, hormonal changes such as pregnancy or menstruation, or certain foods and drugs seem to reactivate the virus.

Genital herpes on the other hand is generally considered to be sexually transmitted. An estimated 40 million people of the world population have genital herpes which makes it a chronic viral infection. About 500,000 people get new symptomatic herpes each year and there are even more people without symptoms. It has been estimated that about 20% of the world population have genital herpes and 90% have oral herpes (cold sores).

To date, there is neither a vaccine to prevent the Herpes infection, nor any way to eliminate the virus from the body. The fact that the herpes virus retreats into the nervous system makes it extremely difficult to eliminate completely.

Previous treatments for herpes virus infections have included the topical application of 5% by weight of acyclovir (Zovirax®), oral administration of valacyclovir HCl (Valtrex®) and laser treatments such as Lectroject®. Each of these treatments is expensive to the patient and the effectiveness is quite slow and often painful. Side effects such as headache and nausea are not uncommon when using repeated applications of acyclovir, whilst the Lectroject® laser method of treating herpes increases the possibility of scar tissue formation.

Accordingly, there remains a need for an effective treatment of skin lesions, and in particular, skin lesions associated with viral infections such as Herpes. Most herpes medications act to “suppress” the virus inside the body in order to reduce outbreaks. In contrast, the present inventors have developed a composition which works by substantially eliminating the virus on direct contact at the outbreak site which accelerates the recovery time of the viral outbreaks and reduces the inconvenience and embarrassment of the condition.

### SUMMARY OF INVENTION

The present invention generally provides topical preparations and methods for treatment of skin and mucosal membrane lesions associated with microbial infections such as Herpes simplex.

In one example of the present invention, there is provided a formulation e.g., an aqueous herbal formulation, comprising an amount of copper effective for topical treatment of one or more skin and mucosal membrane lesions in a subject.

The formulation may be any suitable pharmaceutical formulation capable of being administered by any route e.g., topically, parenterally, by inhalation, ingestion, by suppository, etc. subject to the proviso that such administration is capable of treating or preventing a skin or mucous membrane lesion. Accordingly, the formulation may be a cream, lotion, liquid emulsion, gel, aqueous solution, tablet, or powder.

In the present context, the term “herbal formulation” shall be taken to mean a formulation that does not comprise a synthetic compound as an active agent in the treatment of a lesion of the skin and/or mucosa. For example, a herbal formulation may comprise an active agent type derived from a plant or part thereof or a fungus or a part thereof e.g., from the root, stem, leaf, flower, fruit, or seed of a plant. A herbal formulation may comprise the active agent in a herbal base e.g., a plant-derived base comprising a cream, lotion, liquid emulsion, gel or aqueous solution.

By “amount of copper” is meant a sufficient concentration of copper to ameliorate one or more visible symptoms of a skin lesion such as that associated with infection by a bacterium, fungus or virus such as a Herpes Simplex Virus e.g., HSV-1 or HSV-2, Herpes Zoster Virus, Polio virus, Shingles-associated viruses, Varicella Zoster Virus, Chicken pox-associated viruses or Human Immunodeficiency Virus (HIV-1) or any serotype thereof capable of infecting a human or causing a skin lesion in another animal species. In one example, the copper is present in a trace amount, as an active trace metal.

By “topical treatment” is meant that the formulation is suitable for treatment of a skin or mucous membrane lesion appearing on any one or more body surface(s) such as the skin, hair follicle, nail cuticle, mucous membrane, anus, throat including oral mucosa, eyes including cornea, conjuctiva or eyelid, lips, ears, or genitalia including vagina, labial tissue, penis, scrotum, etc. Again, such treatment does not necessarily require administration by direct contact with that part of the body surface e.g., that part of the body surface having the lesion, however direct topical administration is clearly within the scope of the present invention.

By “lesion” is meant any localized abnormal structural change in a body part such as produced by wounding, infection or other injury involving a cut or break in the skin or mucosa. For example, a lesion in the present context includes any disruption to the skin or mucosa associated with infection of a subject by a bacterium. fungus or virus. For example, lesions associated with virus infection may be apparent on the face and/or mouth (e.g., orofacial herpes), genitalia (e.g., genital herpes), or hands (e.g., herpes whitlow), eye (e.g., herpes keratitis).

In another example, the present invention provides a topical formulation comprising an amount of copper e.g., as an active trace metal, in an aqueous herbal base effective for treatment of skin and mucosal membrane lesions in a subject.

By “topical formulation” is meant that the formulation is suitable for application direct to skin or a mucous membrane, such as directly on a lesion in the skin or mucous membrane. For example, a topical formulation is applied directly to one or more body surface(s) such as the skin, hair follicle, nail cuticle, mucous membrane, anus, throat including oral mucosa, eyes including cornea, conjuctiva or eyelid, lips, ears, or genitalia including vagina, labial tissue, penis, scrotum, etc. Application may comprise direct contact with that part of the body surface e.g., that part of the body surface having the lesion.

By “herbal base” is meant that the composition base into which the active agent is introduced is derived substantially from a non-synthetic source such as a plant or part thereof or a fungus or a part thereof e.g., from the root, stem, leaf, flower, fruit, or seed of a plant. For example, the base may be a cream, lotion, liquid emulsion, gel or aqueous solution. In one particular example, a composition of the present invention may comprising an effective amount of a copper compound in a herbal base comprising an extract selected from the group consisting of: Hypericum perforatum (St. John's wort) extract, Aloe barbadensis extract, Melaleuca alternifolia (tea tree) extract, Melissa officinalis (lemon balm) extract, Prunella vulgaris (selfheal) extract, and combinations thereof. It is to be understood that a herbal extract or herbal base may contribute to efficacy of copper in a formulation of the present invention.

The copper formulations of the present invention will generally comprise a pharmaceutically acceptable copper salt wherein the transition metal cation has a charge of +2 or +3 such as a copper (II) complex or copper (III) complex, and the salt has low toxicity in humans. Examples of pharmaceutically acceptable copper salts are described e.g., Remington's Pharmaceutical Sciences, 18th ed., Mack Publishing, Easton Pa. (1990) and include copper acetate, copper sulphate, copper chloride, copper salicylate, copper nitrate, copper (II)-aspirinate, copper (II) salsalate, a mixed carboxylate copper (II) complex and a caffeine, a copper (II) carboxylate-caffeine complex, and combinations thereof. A solvate of a copper salt may be employed. In one particular example, a formulation according to any example hereof comprises copper in the form of copper sulphate e.g., Cu(II) SO₄ or a solvate thereof. In one example, the copper is provided as Cu(II)SO₄:7H₂O.

A formulation according to any example hereof may comprise copper as an active trace metal. For example, irrespective of the anion employed, the copper ion may be present in the formulation at a concentration in the range of about 1% (w/w) to about 5% (w/w), including about 2% (w/w) or about 3% (w/w) or about 4% (w/w) or about 5% (w/w) or 5.5% (w/w). Alternatively, or in addition, the copper compound is present in the range of between about 5% (w/w) of the formulation and about 9% (w/w) of the formulation, including about 7.0% (w/w) or about 7.1% (w/w) or about 7.2% (w/w) or about 7.3% (w/w) or about 7.4% (w/w) or about 7.5% (w/w) or about 7.6% (w/w) or about 7.7% (w/w) or about 7.8% (w/w) or about 7.9% (w/w) or about 8.0% (w/w) of the formulation. Concentrations of copper ions that are permissible when applied topically may be higher than those concentrations considered toxic when copper is provided to isolated and cultured cells in vitro or by injection.

Pharmaceutically acceptable carriers, excipients and/or diluents utilized should be acceptable for human or veterinary applications. Such carriers, excipients and/or diluents are well-known to those skilled in the art. Carriers and/or diluents suitable for use include any and all solvents, dispersion media, aqueous solutions, coatings, antibacterial and antifungal agents, isotonic and absorption delaying agents, and the like. Except insofar as any conventional media or agent is incompatible with the active ingredient, use thereof in the composition is contemplated. Supplementary active ingredients can also be incorporated into the compositions.

When present, a herbal extract, e.g., selected from the group consisting of: Hypericum perforatum (St. John's wort) extract, Aloe barbadensis extract, Melaleuca alternifolia (tea tree) extract, Melissa officinalis (lemon balm) extract, Prunella vulgaris (selfheal) extract, and combinations thereof, is present at a concentration of about 0.05% (v/v) of an aqueous formulation to about 2% (v/v) of an aqueous formulation, including about 0.05% (v/v) of an aqueous formulation or about 0.1% (v/v) of an aqueous formulation or about 0.15% (v/v) of an aqueous formulation or about 0.2% (v/v) of an aqueous formulation or 0.5% (v/v) of an aqueous formulation or 1.0% (v/v) of an aqueous formulation or 1.5% (v/v) of an aqueous formulation or 2% (v/v) of an aqueous formulation. For example, a herbal extract such as that of Hypericum perforatum or Aloe barbadensis may be obtained or prepared by means of solvent (ethanol) extraction of a plant part e.g., flowers etc.

One or more skin protectants e.g., glycerin may also be included in the formulation e.g., at a concentration of about 1% (w/w) to about 5% (w/w).

One or more preservatives, osmotic regulators, thickeners, flavors, fragrances, emollients, humectants, colorants, pigments and combinations thereof may also be included in the composition of the present invention.

The composition according to any example hereof may further comprise sodium ascorbate e.g., at a concentration of about 3-10% (w/w) and/or hydrogen peroxide e.g., at a concentration of about 3-10% (w/w).

A formulation of the present invention may be administered without limitation in any subject susceptible to a lesion of the skin and/or mucosa, especially subjects that are at risk of virus infection or have a pre-existing infection by one or more viruses. For example, the subject may be a sexually-active person, healthcare worker, or a subject having an immature or suppressed immune system e.g., a newborn, transplant recipient, HIV-1-infected subject or AIDS patient.

The formulations of the present invention are effective in ameliorating one or more visible symptoms of a skin lesion or lesion in the mucosa e.g., associated with infection. For example, the formulation will reduce duration and/or severity of a lesion and/or reduce pain and/or reduce inflammation and/or reduce itchiness and/or reduce virus shed. Alternatively, or in addition, a formulation of the present invention prevents or assists in preventing a lesion from increasing in size and/or decrease virus shed or spread during healing. Alternatively, or in addition, a formulation of the present invention prevents or reduces recurrence of a lesion or virus outbreak.

A formulation of the present invention may be applied at any stage during presentation of symptoms in a subject. For example, a formulation of the present invention provides a beneficial effect when applied early e.g., at first appearance of a lesion or with 24 hours or 48 hours or 72 hours of the first appearance of a lesion or before the lesion weeps. Alternatively, the formulation may be applied to an active lesion.

In one example, the present invention is performed as a stand-alone therapy. In another example, the present invention is performed as an adjunct therapy with other prophylactic or therapeutic treatment e.g., one or more of an antiviral agent, non-steroidal anti-inflammatory drug (NSAID), vaccine, dietary supplement, etc. including one or more of acyclovir (Zovirax), valaciclovir (Valtrex), famciclovir (Famvir), penciclovir, docosanol, tromantadine, zilactin, lipactin, tea tree oil, cimetidine, probenecid, aspirin, lidocain, prilocaine, tetracaine, petroleum jelly, Herpevac, lysine, lactoferrin, vitamin C, vitamin A, vitamin E, and zinc.

In another example of the present invention, there is provided a method of treating or preventing one or more skin lesions said method comprising applying to the lesion a therapeutically effective amount of a formulation according to any example hereof e.g., for a time and under conditions sufficient for ameliorate one or more visible symptoms of a skin lesion or lesion in the mucosa e.g., associated with infection. For example, the amelioration of one or more symptoms may comprise a reduced duration and/or severity of a lesion and/or reduces pain and/or reduced inflammation and/or reduced itchiness and/or reduced virus shed and/or decrease in virus shed and/or decrease in virus spread. Alternatively, or in addition, a formulation of the present invention is applied for a time and under conditions sufficient to prevent or reduce recurrence and/or to prevent a lesion from increasing in size.

The therapeutic or prophylactic method of the present invention is particularly suited to the treatment and prevention of skin and/or mucosal lesions associated with infection by a bacterium, fungus or virus, including one or more of Herpes Simplex Virus e.g., HSV-1 or HSV-2, Herpes Zoster Virus, Polio virus, Shingles-associated viruses, Varicella Zoster Virus, Chicken pox-associated viruses, Human Immunodeficiency Virus (HIV-1), or any serotype thereof capable of infecting a human or causing a skin lesion in another animal species. In a particular example, the present invention provides a method for the treatment and/or prevention of skin lesions associated with infection by a herpesvirus e.g., HSV-1 and/or HSV-2 or any serotype thereof capable of infecting a human or causing a skin lesion in another animal species.

A formulation according to any example hereof may be applied once or more times during an outbreak, such as prior to virus shed or weeping of the lesion or to a visible and/or mature lesion. For example, a lesion may be applied to the mouth area of a subject suffering from a cold sore, or around the genitalia of a subject suffering from genital herpes.

Another example of the present invention provides for use of a formulation according to any example hereof in the manufacture of a medicament for the treatment or prevention of a virus-associated skin or mucosal membrane lesion e.g., wherein the virus is selected from the group consisting of Herpes Simplex Virus e.g., HSV-1 or HSV-2, Herpes Zoster Virus, Polio virus, Shingles-associated viruses, Varicella Zoster Virus, Chicken pox-associated viruses, Human Immunodeficiency Virus (HIV-1), or any serotype thereof capable of infecting a human or causing a skin lesion in another animal species, especially HSV-1 and/or HSV-2 or any serotype thereof capable of infecting a human or causing a skin lesion in another animal species.

A further example of the present invention provides a process of producing a composition for the treatment of viral associated skin and mucosal membrane lesions, the process comprising forming a copper solution from a copper compound, adding glycerin to the copper solution, adding a herbal extract to the copper solution, bringing the solution to a desired concentration using water, optionally adding a preservative; and optionally filtering to remove any sediment.

Throughout this specification including the claims, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of the invention disclosed in this application.

The present invention is also described with reference to the following examples. It will be appreciated by persons skilled in the art that numerous variations and/or modifications may be made to the invention as shown in the specific examples without departing from the spirit or scope of the invention as broadly described. The present examples are, therefore, to be considered in all respects as illustrative and not restrictive.

### MODE(S) FOR CARRYING OUT THE INVENTION

Previous topical medications have used high concentrations of copper sulphate, some as high as 10% by weight. High concentrations of copper have been found to cause blood poisoning which can be potentially fatal. In contrast, the present inventors have surprisingly shown that a copper compound used at a concentration of 5-9% by weight in combination with 0.05 to 0.15% by weight of hypericum perforatum extract provides a synergistic antiviral effect in the treatment of skin lesions. Use of a lower concentration of copper results in the composition being safe, providing an effective dose to be used topically, even on open wounds. Prior art uses indicate that it is necessary to use high concentrations of copper sulphate to provide adequate anti-viral activity.

The present inventors have found that lower concentrations of a copper salt in a herbal base e.g., Hypericum perforatum extract is significantly effective in the treatment of a lesion of the skin or mucosa, especially those caused by viral agents such as HSV-1 and/or HSV-2. More particularly, a composition comprising copper ions, such as in the form of copper sulphate pentahydrate and a herbal base such as Hypericum perforatum extract or Aloe barabensis extract is effective in dramatically reducing the healing time of lesions due to infection by herpesviruses, stopping the normal progression of the viral outbreak from the stage at which the initial outbreak occurred, and also in reducing recurrence of lesions. The composition of the present invention is a copper-based solution with copper as the active trace metal in an aqueous herbal base.

Whilst it is not intended that the present invention should be restricted in any way by a theoretical explanation of the mode of action of copper, it is believed that copper may exert an antiviral and healing effect by virtue of the composition being slightly acidic, which is corrosive to the virus. Accordingly, preferred compositions possess an acid group and sulfur molecule to assists in penetration of the composition through the protective capsule of a virus such as herpesvirus, thereby allowing for direct contact with the virus and subsequent destruction of the virus.

In one example, the compositions of the present invention are those recognised in the pharmaceutical arts as being suitable for topical application and include, without intended limitation, creams, lotions, liquid emulsions, gels, aqueous solutions and the like. The present compositions preferably include copper sulphate pentahydrate in from about 5 to 9, preferably 7.8% by weight or copper ion from about 1 to 5%, (equivalent to about 3 to 7% copper sulphate), preferably 5% by weight.

Optionally, a skin protectant e.g., glycerin, is included e.g., from about 5% by weight, and a herbal extract is included from about 0.05 to 0.15%, preferably about 0.1% by weight. In addition to the enhanced therapeutic effect, the subject composition is also advantageous in that it is safe and has no known side-effects. The composition can also be safely used for veterinary purposes. A skin protectant forms a barrier over the skin surface to protect against irritation due to touching, rubbing etc. The skin protectant also provides a protective barrier over the lesion, preventing loss of the active ingredient to the action of saliva. In a preferred embodiment, the skin protectant is in the form of glycerin.

In addition to the foregoing ingredients, the composition of the present invention may contain other ingredients such as are recognised by those skilled in the pharmaceutical industry as being typically present in such formulations. These include, without limitation, one or more preservatives, osmotic regulators, thickeners, flavours, fragrances, emollients, humectants, colorants, pigments and the like. It would be clear to the skilled addressee that the compounding of the composition of the present invention will be carried out utilising some or all of these ingredients depending on the area of application and intended use. For example, for a preparation intended for application in or around the mouth, it may be necessary to add flavours to mask the taste of the essential ingredients.

Although best long term results are achieved by applying the composition to a visible and mature lesion, patients have reported great success in preventing outbreaks by applying the composition in the early stages. This can include applying the composition topically to the affected area as the first sign of symptoms such as itching, tingling, redness or inflammation.

### Example 1

### Exemplary Copper Formulations of the Invention in Aqueous Herbal Base

An aqueous solution was prepared from formula 1 given below.

### Formula 1

| Components | Typical Amount | |
|---|---|---|
| Assay as copper sulphate | 7.80% | w/w max |
| pentahydrate (CuSO₄—SH₂O) | | |
| (Assay equivalent copper sulphate | 5.0% | w/w max |
| anhydrous (CuSO₄) | | |
| Glycerin (Glycerol) | 5.0% | w/w max |
| Hypericum perforatum | 0.1% | v/v max |
| Germall Plus (preservative) | 0.3% | v/v max |

| | |
|---|---|
| Water Purified | Balance |

### Formula 2

| Components | Typical Amount | |
|---|---|---|
| Assay as copper sulphate | 9% | w/w max |
| Glycerin (Glycerol) | 5% | w/w max |
| Hypericum perforatum | 0.15% | v/v max |
| Germall Plus (preservative) | 0.3% | v/v max |

| | |
|---|---|
| Water Purified | Balance |

### Formula 3

| Components | Typical Amount | |
|---|---|---|
| Assay as copper sulphate | 5% | w/w max |
| Glycerin (Glycerol) | 1% | w/w max |
| Hypericum perforatum | 0.05% | v/v max |
| Germall Plus (preservative) | 0.1% | v/v max |

| | |
|---|---|
| Water Purified | Balance |

### Formula 4

| Components | Typical Amount | |
|---|---|---|
| Assay as copper chloride | 9% | w/w max |
| Glycerin (Glycerol) | 3.0% | w/w max |
| Hypericum perforatum | 0.2% | v/v max |
| Germall Plus (preservative) | 0.2% | v/v max |

| | |
|---|---|
| Water Purified | Balance |

### Formula 5

| Components | Typical Amount | |
|---|---|---|
| Assay as copper chloride | 9% | w/w max |
| Glycerin (Glycerol) | 3.0% | w/w max |
| Hypericum perforatum | 0.2% | v/v max |
| Germall Plus (preservative) | 0.2% | v/v max |

| | |
|---|---|
| Water Purified | Balance |

### Formula 6

| Components | Typical Amount | |
|---|---|---|
| Assay as copper salicylate | 9% | w/w max |
| Glycerin (Glycerol) | 3.0% | w/w max |
| Hypericum perforatum | 0.2% | v/v max |
| Germall Plus (preservative) | 0.2% | v/v max |

| | |
|---|---|
| Water Purified | Balance |

Each solution was prepared by filling a suitable vessel with about 60% distilled water. Copper compound was added to water with continuous stirring. Mixing of a solution continued for 10 minutes or until the copper was fully dissolved. Glycerin was added to a solution and mixed for a few minutes. Hypericum perforatum was gradually added with continuous mixing. Each solution was then brought to final weight by the slow addition of the required amount of water with continuous blending for about 10 minutes. Preservative Germall Plus was added and a solution was then allowed to stand for 12 to 15 hours to stabilise. Each solution was then filtered to remove the sediment and packaged.

### Example 2

### Treatment of Herpes Simplex Patients with a Copper Formulation of the Invention

A solution prepared in Example 1 was tested for its effectiveness against Herpes virus. To determine efficacy of the composition in reducing the healing time of lesions associated with Herpes virus infection and/or reducing the recurrence of the lesions, 51 patients were observed.

Of the 51 patients treated with the composition, 34 suffered from mouth lesions associated with Herpes Simplex virus 1 infection, and 19 suffered from genital lesions associated with Herpes Simplex virus 2 infection, and 2 patients suffered from both mouth and genital lesions. Following topical application of the composition to the area of the viral-associated lesion, 38 of the 51 patients reported a dramatic reduction in the healing time of the lesion (with scab formation occurring within 24 hours) and 47 of the 51 patients reported a reduced recurrence of lesions. All patients reported a substantial reduction in pain and discomfort associated with the lesions following application of the composition.

### Example 3

### Safety, Tolerability and Efficacy of the Copper Formulation in Patients Suffering from HSV-1 and/or HSV-2

A trial enrolled 150 herpes simplex patients (HSV 1 & HSV 2) having active lesions on external genitalia and skin, aged between 18 and 55 years.

Subjects were randomized into two groups (A and B). Subjects in Group A topically applied a composition as defined in Example 1, transferring 2-4 drops of aqueous copper formulation (depending upon the affected area) to a wet cotton swab (enough to saturate it) on the affected part only once at clinic. Subjects in Group B topically applied 0.5-1.5 grams of the comparator article (acyclovir 5% cream) twice daily (once in the morning and once at night) to cover affected areas for 7 days. Treatments were initiated on Day 1, pending the results of laboratory investigations. Patients in Group A presented for efficacy & safety assessments on Day 2, Day 3, Day 8 and Day 14. Group B patients presented on Day 3 and Day 8 for efficacy evaluation and on Day 14 for follow up. Patients also recorded their self-assessment of symptoms in a patient diary from Day 1 to Day 14.

Safety was evaluated by determining adverse event reports throughout the study. Hematology and clinical chemistry labs, urinalysis, and physical exam of basic systems were obtained at initial screening and Day 14 (end of study) visits. The patient was withdrawn immediately from the study if the results show abnormal values in laboratory investigation reports.

Efficacy endpoint assessments determine time to achieve greater than about 50% crusting/scabbing or healed ulcer. Cutaneous assessments are performed to determine disappearance of erythema, crust/scab formation in ulcers, disappearance of pain and disappearance of itching and burning sensation.

Cutaneous efficacy assessments are performed at each visit on Day 2, Day 3 and Day 8 in Group A and on Day 3 and Day 8 or until 100% crusting is observed in Group B.

Local cutaneous tolerability is evaluated with assessments of erythema, induration and stinging sensation on Day 1, Day 2, Day 3 and Day 8 in Group A and on Day 3 and Day 8 in Group B or until 100% crusting is observed.

The foregoing case studies demonstrate the efficacy of copper formulations in a herbal base for the treatment of skin and mucosal membrane lesions associated with herpesvirus infection.

The claims defining the invention are as follows:

1. An aqueous herbal formulation comprising an amount of copper as an active trace metal effective for topical treatment of skin and mucosal membrane lesions in a subject.

2. A topical formulation comprising an amount of copper as an active trace metal in an aqueous herbal base effective for treatment of skin and mucosal membrane lesions in a subject.

3. The formulation of claim 2, wherein the base is a cream, lotion, liquid emulsion, gel or aqueous solution.

4. The formulation according to any one of claims 1 to 3, wherein the copper as an active trace metal is in the form of copper sulphate.

5. The formulation according to any one of claims 1 to 4, wherein the copper as an active trace metal is at a copper ion concentration of about 1 to 5% w/w.

6. The formulation according to claim 5, wherein the copper ion concentration is 5% w/w.

7. A composition for topical treatment of skin and mucosal membrane lesions comprising an effective amount of:
a copper compound;
and hypericum perforatum extract.

8. The composition according to claim 7, wherein the copper compound is copper sulphate, copper chloride or copper salicylate.

9. The composition according to claim 8, wherein the copper compound is copper sulphate.

10. The composition according to claim 9 or the formulation according to claim 4, wherein the copper sulphate is copper sulphate pentahydrate anhydrous.

11. The composition according to any one of claims 7 to 10, wherein the copper compound is at a concentration of about 5-9% (w/w).

12. The formulation according to claim 4 or the composition according to claim 9 or 10, wherein the copper sulphate is provided at a concentration of about 7.8% (w/w).

13. The composition according to any one of claims 7 to 12, wherein the copper compound provides a copper ion concentration of about 1-5% (w/w).

14. The composition according to any one of claims 7 to 13, wherein the hypericum perforatum extract is at a concentration of about 0.05 to 0.15% (v/v).

15. The formulation according to any one of claim 1 to 6, 10 or 12, or the composition according to any one of claims 7 to 14, or further comprising:
a skin protectant.

16. The formulation of claim 15 or the composition of claim 15, wherein the skin protectant is at a concentration of about 1 to 5% (w/w).

17. The formulation of claim 15 or 16, or the composition of claim 15 or 16, wherein the skin protectant is glycerin.

18. The formulation according to any one of claims 1 to 6, 10, 12, or 15 to 17, or the composition according to any one of claims 7 to 15, further comprising any one or more of a preservative, osmotic regulator, thickener, flavor, fragrance, emollient, humectant, colorant, or pigment.

19. The formulation of claim 18, or the composition of claim 18, wherein the preservative is Germall Plus.

20. The formulation of claim 18 or 19, or the composition of claim 18 or 19, comprising about 0.1 to about 0.3% (w/w) preservative.

21. The composition according to any one of claims 7 to 20, further comprising aloe vera at a concentration of about 1.0% (v/v).

22. The composition according to any one of claims 7 to 21, further comprising sodium ascorbate at a concentration of about 3-10% (w/w).

23. The composition according any one of claims 7 to 22, further comprising hydrogen peroxide at a concentration of about 3-10% (w/w).

24. The composition according to any one of claims 7 to 23 in the form of a cream, lotion, emollient, gel or emulsion.

25. A method of treating or preventing skin lesions comprising applying to the lesion a therapeutically effective amount of a formulation according to any one of claims 1 to 6, 10, 12, or 15 to 20, or the composition according to any one of claims 7 to 24.

26. The method of claim 25, wherein the lesion is a skin or mucosal membrane lesion.

27. The method of claim 26, wherein the skin lesion is associated with a bacterial, fungal or viral infection.

28. The method of claim 27, wherein the viral infection is selected from the group consisting of Herpes Simplex virus, Herpes Zoster virus, Polio virus, Shingles-associated viruses, Varicella Zoster virus, Chicken pox-associated viruses or Human Immunodeficiency virus.

29. The method of claim 28, wherein the viral infection is caused by Herpes Simplex virus.

30. Use of the formulation according to any one of claims 1 to 6, 10, 12, or 15 to 20, or the composition according to any one of claims 7 to 24 in the manufacture of a medicament for the treatment or prevention of viral associated skin or mucosal membrane lesions.

31. The use of claim 30, wherein the skin lesion is associated with a bacterial, fungal or viral infection.

32. The use of claim 31, wherein the viral infection is selected from the group consisting of Herpes Simplex virus, Herpes Zoster virus, Polio virus, Shingles-associated viruses, Varicella Zoster virus, Chicken pox-associated viruses or Human Immunodeficiency virus.

33. The use of claim 32, wherein the viral infection is caused by Herpes Simplex virus.

34. A process of producing a composition for the treatment of viral associated skin and mucosal membrane lesions, the process comprising:
forming a copper solution from a copper compound;
adding glycerin to the copper solution;
adding hypericum perforatum to the copper solution;
bringing the solution to a desired concentration using water;
optionally adding a preservative; and
optionally filtering to remove any sediment.
